(19) United States
(12) Patent Application Publication
(10) Pub. No.: US 2024/0201189 A1
(43) Pub. Date: Jun. 20, 2024

(54) FLUORESCENT PROBE FOR USE IN DETECTION OF PANCREATIC CANCER

(71) Applicant: The University of Tokyo, Tokyo (JP)

(72) Inventors: Yasuteru URANO, Tokyo (JP); Mako KAMIYA, Kanagawa (JP); Kiyoshi HASEGAWA, Tokyo (JP); Takeaki ISHIZAWA, Osaka (JP); Ryugen TAKAHASHI, Tokyo (JP)

(73) Assignee: The University of Tokyo, Tokyo (JP)

(21) Appl. No.: 18/286,291

(22) PCT Filed: Apr. 12, 2022
(86) PCT No.: PCT/JP2022/017566

§ 371 Date: Oct. 10, 2023

Related U.S. Application Data
(60) Provisional application No. 63/173,574, filed on Apr. 12, 2021.

(30) Foreign Application Priority Data
May 13, 2021 (JP) 2021-081777

(51) Int. Cl. G01N 33/574; C07K 5/062; C07K 5/065; C07K 5/072; C07K 5/078

(52) U.S. Cl. CPC G01N 33/57438; C07K 5/06026; C07K 5/06078; C07K 5/06113; C07K 5/06165; G01N 33/57492

(57) ABSTRACT

[Problem] To provide a fluorescent probe capable of detecting pancreatic cancer specifically.

[Solution] A fluorescent probe for use in the detection of pancreatic cancer, which comprises a compound represented by general formula (I) or a salt thereof.

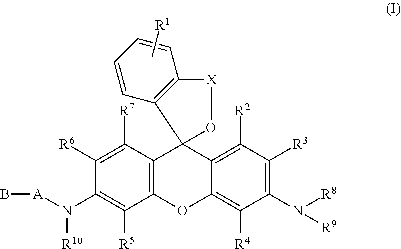

### TECHNICAL FIELD

The present invention relates to a fluorescent probe for detecting pancreatic cancer and a method for detecting a pancreatic cancer cell or cancer tissue using the same.

### BACKGROUND ART

Pancreatic cancer is one of the major life-threatening diseases (for example, Non Patent Literature 1). Despite recent advances in chemotherapy and radiotherapy, complete resection of carcinomatous tissue remains central to curative treatment (Non Patent Literatures 2 and 3).

However, it is often difficult to accurately identify boundaries of cancer spread during surgery, which may lead to incomplete removal of carcinomatous tissue and unfavorable postoperative survival (Non Patent Literatures 2 and 3). In particular, in a patient receiving preoperative chemotherapy (Non Patent Literature 4), identification of viable carcinomatous tissue may be more difficult even by pathological examination of a resected specimen (Non Patent Literature 5).

In 2011, Urano, one of the present inventors, and co-workers reported a new fluorescence imaging technique using an activatable type probe. The probe does not initially emit fluorescence, but emits a visible fluorescence signal immediately after hydrolysis by γ-glutamyl transpeptidase overexpressed in cancer cells (Non Patent Literature 6). Since then, 400 or more activatable fluorescent probes formed of an amino acid or glucose and a fluorescent substance such as hydroxymethyl rhodamine green (HMRG) have been developed (Non Patent Literatures 7 and 8), which allows visualization of breast cancer, esophageal cancer, liver cancer, lung cancer, head and neck cancer, colorectal cancer, and thyroid cancer.

However, fluorescent probes capable of specifically detecting pancreatic cancer have not yet been developed.

### CITATION LIST

### Non Patent Literature

Non Patent Literature 1: Hidalgo M. Pancreatic cancer. N Engl J Med. 2010 Apr 29; 362 (17): 1605-17.

Non Patent Literature 2: Demir IE, Jager C, Schlitter AM, Konukiewitz B, Stecher L, Schorn S, et al. R0Versus R1 Resection Matters after Pancreaticoduodenectomy, and Less after Distal or Total Pancreatectomy for Pancreatic Cancer. Ann Surg. 2018 Dec; 268 (6): 1058-1068.

Non Patent Literature 3: Tummers WS, Groen JV, Sibinga Mulder BG, Farina-Sarasqueta A, Morreau J, Putter H, et al. Impact of resection margin status on recurrence and survival in pancreatic cancer surgery. Br J Surg. 2019 Jul; 106(8): 1055-1065.

Non Patent Literature 4: Gillen S, Schuster T, Meyer Zum Buschenfelde C, Friess H, Kleeff J.

Preoperative/neoadjuvant therapy in pancreatic cancer: a systematic review and meta-analysis of response and resection percentages. PLOS Med. 2010 Apr 20; 7 (4): e1000267.

Non Patent Literature 5: Verbeke C, Lohr M, Karlsson JS, Del Chiaro M. Pathology reporting of pancreatic cancer following neoadjuvant therapy: challenges and uncertainties. Cancer Treat Rev. 2015 Jan; 41(1): 17-26.

Non Patent Literature 6: Urano Y, Sakabe M, Kosaka N, Ogawa M, Mitsunaga M, Asanuma D, et al. Rapid cancer detection by topically spraying a γ-glutamyltranspeptidaseactivated fluorescent probe. Sci Transl Med. 2011 Nov 23; 3 (110): 110ra119.

Non Patent Literature 7: Fujita K, Kamiya M, Yoshioka T, Ogasawara A, Hino R, Kojima R, et al. Rapid and Accurate Visualization of Breast Tumors with a Fluorescent Probe Targeting α-Mannosidase 2C1. ACS Cent Sci. 2020 Dec 23; 6 (12): 2217-2227.

Non Patent Literature 8: Kuriki et al. in press

### SUMMARY OF INVENTION

### Technical Problem

An object of the present invention is to provide a fluorescent probe capable of specifically detecting pancreatic cancer. In addition, an object of the present invention is to provide a method for detecting a pancreatic cancer cell or cancer tissue using the fluorescent probe.

### Solution to Problem

As a result of preparing a lysate by collecting a carcinomatous tissue fragment and a non-carcinomatous pancreatic tissue fragment from a pancreatic cancer resected specimen and searching for a fluorescent probe capable of specifically detecting pancreatic cancer using a library of enzyme probes including approximately 400 types of HMRG derivative probes, the present inventors have found that an HMRG derivative probe having a specific structure can specifically detect pancreatic cancer, thereby completing the present invention.

That is, the present invention provides:
[1] A fluorescent probe for detecting pancreatic cancer, the fluorescent probe comprising a compound represented by the following General Formula (I) or a salt thereof,

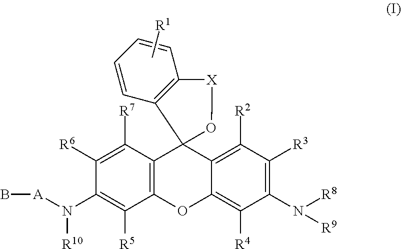

(in the formula,

R¹ represents a hydrogen atom or one to four identical or different substituents bonded to a benzene ring;

R², R³, R⁴, R⁵, R⁶, and R⁷ each independently represent a hydrogen atom, a hydroxyl group, an alkyl group, or a halogen atom;

R⁸, R⁹, and R¹⁰ each independently represent a hydrogen atom or an alkyl group;

X represents a C₁-C₃ alkylene group;

A is a proline residue; and

B is an amino acid residue selected from a glycine residue, a leucine residue, a proline residue, a tyrosine residue, and an N^{α}-acetyl-lysine residue;

where A is linked to adjacent NH in the formula by forming an amide bond, and B is linked to A by forming an amide bond);
[2] The fluorescent probe according to [1], in which B is a glycine residue;
[3] The fluorescent probe according to [1] or [2], in which R¹, R², R³, R⁴, R⁵, R⁶, R⁷, R⁸, R⁹, and R¹⁰ are hydrogen atoms, and X is a methylene group;
[4] A fluorescent probe for detecting pancreatic cancer, the fluorescent probe containing a compound selected from the following group or a salt thereof;

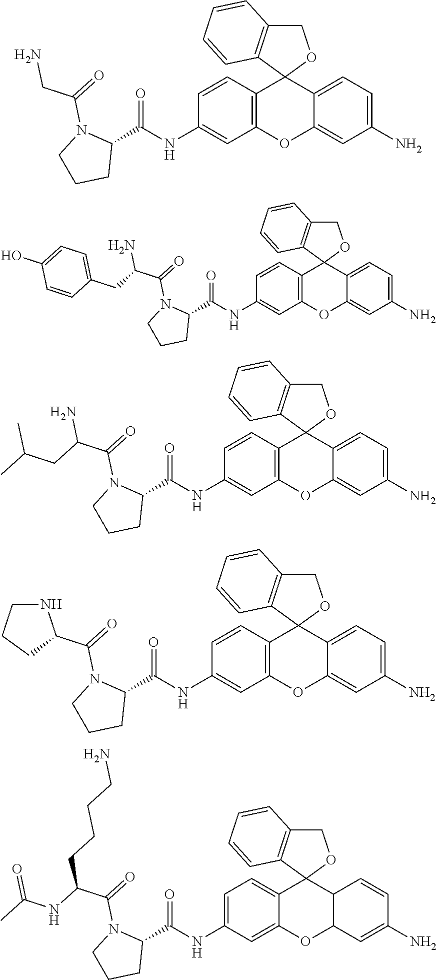

[5] A composition for detecting pancreatic cancer, the composition comprising the fluorescent probe according to any one of [1] to [4];
[6] A kit for detecting pancreatic cancer, the kit comprising the fluorescent probe according to any one of [1] to [4];
[7] A composition for diagnosing pancreatic cancer, the composition comprising the fluorescent probe according to any one of [1] to [4];

[8] The composition for diagnosing pancreatic cancer according to [7], in which the composition is used in a cancer surgical treatment or a cancer examination;
[9] The composition for diagnosing pancreatic cancer according to [8], in which the cancer surgical treatment is open surgery or endoscopic surgery;
[10] A method for detecting a pancreatic cancer cell or cancer tissue, the method comprising the steps of:

applying the fluorescent probe according to any one of [1] to [4] to tissue collected from a pancreas of a subject;

irradiating the tissue after the application with excitation light; and

detecting fluorescence from the tissue;
[11] A method for sensing pancreatic cancer, the method comprising: (a) a step of applying the fluorescent probe according to any one of [1] to [4] to a clinical specimen of pancreatic cancer; and (b) measuring a fluorescence image of the clinical specimen of pancreatic cancer to which the fluorescent probe is applied;
[12] The method according to [11], further including visualizing the fluorescence image using a fluorescence imaging means;
[13] A method for determining the presence of a pancreatic cancer cell in a subject and/or identifying a range of pancreatic cancer tissue, the method comprising: (a) a step of applying a fluorescent probe comprising a compound represented by the following General Formula (I) or a salt thereof to a specimen surgically resected from a pancreas of the subject; and (b) measuring a fluorescence image of the resected specimen to which the fluorescent probe is applied,

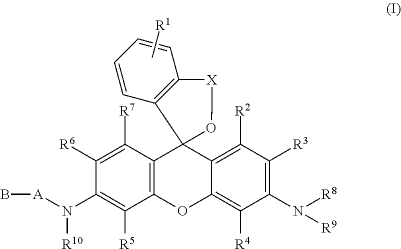

(in the formula,

R¹ represents a hydrogen atom or one to four identical or different substituents bonded to a benzene ring;

R², R³, R⁴, R⁵, R⁶, and R⁷ each independently represent a hydrogen atom, a hydroxyl group, an alkyl group, or a halogen atom;

R⁸, R⁹, and R¹⁰ each independently represent a hydrogen atom or an alkyl group;

X represents a C₁-C₃ alkylene group;

A is a proline residue; and

B is an amino acid residue selected from a glycine residue, a leucine residue, a proline residue, a tyrosine residue, and an N^{α}-acetyl-lysine residue;

where A is linked to adjacent NH in the formula by forming an amide bond, and B is linked to A by forming an amide bond);
[14] The method according to [13], in which B is a glycine residue;
[15] The method according to or [14], in which R¹, R², R³, R⁴, R⁵, R⁶, R⁷, R⁸, R⁹, and R¹⁰ are hydrogen atoms, and X is a methylene group;
[16] The method according to any one of to [15], further including visualizing the fluorescence image using a fluorescence imaging means;
[17] The method according to any one of to [16], in which the method is performed during a surgical treatment of pancreatic cancer; and
[18] The method according to [17], in which the surgical treatment of pancreatic cancer is open surgery or endoscopic surgery.

### Advantageous Effects of Invention

According to the present invention, it is possible to provide a fluorescent probe capable of specifically detecting pancreatic cancer. In addition, it is possible to provide a method for detecting a pancreatic cancer cell or cancer tissue using the fluorescent probe of the present invention.

It is possible to identify viable pancreatic cancer tissue in a human resected specimen in real time by the fluorescent probe for detecting pancreatic cancer of the present invention.

In addition, it is possible to provide a method for determining the presence of a pancreatic cancer cell in a subject and/or a method for identifying a range of pancreatic cancer tissue during a surgical treatment of pancreatic cancer using the fluorescent probe of the present invention. Therefore, it is possible to distinguish carcinomatous tissue from surrounding non-carcinomatous tissue as a fluorescent region in a specimen surgically resected from a pancreas of a subject.

In addition, in a patient who received preoperative chemotherapy, cancer invasion that cannot be confirmed by the naked eye may occur around a blood vessel such as the splenic artery, but when the identification method of the present invention is used, it is possible to visualize cancer invasion that cannot be confirmed by the naked eye around a blood vessel such as the splenic artery by fluorescence imaging, which makes it possible to reduce the amount of cancer left during surgery.

### BRIEF DESCRIPTION OF DRAWINGS

FIG. 1A illustrates an increase in FI of candidate probes in primary selection using a lysate obtained from a pancreatic cancer patient.

FIG. 1B illustrates a TBR based on the increase in FI at 30 minutes after administration of 5 types of probes to tissue fragments.

FIG. 1C illustrates a tendency of the increase in FI in carcinomatous and non-carcinomatous tissue fragments after administration of GP-HMRG.

FIG. 2 illustrates fluorescence imaging (Patient No. 2) on all surgical specimens using GP-HMRG showing a uniform increase in fluorescence signal in pancreatic cancer tissue.

FIG. 3 illustrates fluorescence imaging (Patient No. 6) on all surgical specimens using GP-HMRG showing a non-uniform increase in fluorescence signal in pancreatic cancer tissue.

FIG. 4 illustrates fluorescence imaging (Patient No. 8) on all surgical specimens using GP-HMRG showing cancer invasion into the splenic artery.

FIG. 5 illustrates a temporal change in fluorescence intensity of GP-HMRG when each enzyme is added.

### MODE FOR CARRYING OUT THE INVENTION

### 1. Definition

In the present specification, the “halogen atom” means a fluorine atom, a chlorine atom, a bromine atom, or an iodine atom.

In the present specification, the “alkyl” may be any of linear alkyl, branched alkyl, cyclic alkyl, or an aliphatic hydrocarbon group composed of a combination thereof. The number of carbon atoms in the alkyl group is not particularly limited, and is, for example, 1 to 20 carbon atoms (C₁₋₂₀), 3 to 15 carbon atoms (C₃₋₁₅) , or 5 to 10 carbon atoms (C₅₋₁₀). In a case where the number of carbon atoms is specified, it means “alkyl” having the number of carbon atoms in the range of the number. For example, C₁₋₈ alkyl includes methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. In the present specification, the alkyl group may have one or more arbitrary substituents. Examples of the substituent include an alkoxy group, a halogen atom, an amino group, a mono- or disubstituted amino group, a substituted silyl group, and acyl, but are not limited thereto. In a case where the alkyl group has two or more substituents, these substituents may be the same as or different from each other. The same applies to alkyl moieties of other substituents including alkyl moieties (for example, an alkoxy group, an arylalkyl group, and the like).

In the present specification, in a case where a certain functional group is defined as “which may have a substituent”, the type of substituent, a substitution position, and the number of substituents are not particularly limited, and in a case where a certain functional group has two or more substituents, these substituents may be the same as or different from each other. Examples of the substituent include an alkyl group, an alkoxy group, a hydroxyl group, a carboxyl group, a halogen atom, a sulfo group, an amino group, an alkoxycarbonyl group, and an oxo group, but are not limited thereto. Substituents may be further present in these substituents. Examples thereof include a halogenated alkyl group and a dialkylamino group, but are not limited thereto.

In the present specification, the “alkoxy group” is a structure in which the alkyl group is bonded to an oxygen atom, and examples thereof include a linear alkoxy group, a branched alkoxy group, a cyclic alkoxy group, and a saturated alkoxy group composed of a combination thereof. Preferred examples thereof include a methoxy group, an ethoxy group, an n-propoxy group, an isopropoxy group, a cyclopropoxy group, an n-butoxy group, an isobutoxy group, an s-butoxy group, a t-butoxy group, a cyclobutoxy group, a cyclopropylmethoxy group, an n-pentyloxy group, a cyclopentyloxy group, a cyclopropylethyloxy group, a cyclobutylmethyloxy group, an n-hexyloxy group, a cyclohexyloxy group, a cyclopropylpropyloxy group, a cyclobutylethyloxy group, and a cyclopentylmethyloxy group.

In the present specification, “alkylamino” and “arylamino” mean an amino group in which a hydrogen atom of a —NH₂ group is substituted with one or two of the alkyl or aryl. Examples thereof include methylamino, dimethylamino, ethylamino, diethylamino, ethylmethylamino, and benzylamino. Similarly, “alkylthio” and “arylthio” mean a group in which a hydrogen atom of a —SH group is substituted with the alkyl or aryl. Examples thereof include methylthio, ethylthio, and benzylthio.

“Amide” used in the present specification includes both RNR′CO— (when R=alkyl, alkylaminocarbonyl-) and RCONR′— (when R=alkyl, alkylcarbonylamino-).

In the present specification, the term “ring structure” when formed by a combination of two substituents means a heterocyclic or carbocyclic group, and such a group can be saturated, unsaturated, or aromatic. Therefore, the ring structure includes cycloalkyl, cycloalkenyl, aryl, and heteroaryl as defined above. Examples thereof include cycloalkyl, phenyl, naphthyl, morpholinyl, piperdinyl, imidazolyl, pyrrolidinyl, and pyridyl. In the present specification, a substituent can form a ring structure with another substituent, and in a case where such substituents are bonded to each other, those skilled in the art can understand that a specific substitution, for example, a bond to hydrogen is formed. Therefore, in a case where it is described that specific substituents together form a ring structure, those skilled in the art can understand that the ring structure can be formed by a usual chemical reaction and is easily generated. Such ring structures and a formation process thereof are all within the purview of those skilled in the art.

### 2. Fluorescent Probe for Detecting Pancreatic Cancer

One embodiment of the present invention is a fluorescent probe for detecting pancreatic cancer that comprises a compound represented by the following General Formula (I) or a salt thereof (hereinafter, also referred to as a “fluorescent probe of the present invention”).

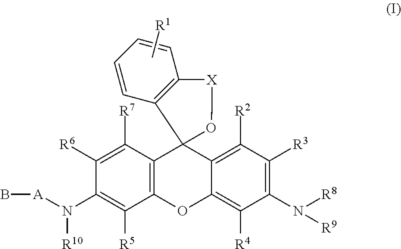

In General Formula (I), R¹ represents a hydrogen atom or one to four substituents bonded to a benzene ring. Examples of the substituent include an alkyl group, an alkoxy group, a halogen atom, an amino group, a mono- or disubstituted amino group, a substituted silyl group, and an acyl group, but are not limited thereto. In a case where the benzene ring has two or more substituents, these substituents may be the same as or different from each other. R¹ is preferably a hydrogen atom.

R², R³, R⁴, R⁵, R⁶, and R⁷ each independently represent a hydrogen atom, a hydroxyl group, an alkyl group, or a halogen atom. It is preferable that R² and R⁷ are hydrogen atoms. In addition, it is preferable that R³, R⁴, R⁵, and R⁶ are hydrogen atoms. It is further preferable that all of R², R³, R⁴, R⁵, R⁶, and R⁷ are hydrogen atoms.

R⁸, R⁹, and R¹⁰ each independently represent a hydrogen atom or an alkyl group. In a case where both R⁸ and R⁹ represent an alkyl group, the alkyl groups may be the same as or different from each other. For example, in a case where both R⁸ and R⁹ are hydrogen atoms, it is preferable that R⁸ is an alkyl group and R⁹ is a hydrogen atom, and it is further preferable that both R⁸ and R⁹ are hydrogen atoms.

In addition, it is preferable that R¹⁰ is a hydrogen atom.

X represents a C₁-C₃ alkylene group. The alkylene group may be either a linear alkylene group or a branched alkylene group. For example, in addition to a methylene group (—CH₂—), an ethylene group (—CH₂—CH₂—), and a propylene group (—CH₂—CH₂—CH₂—), —CH (CH₃)—, —CH₂—CH (CH₃)—, —CH (CH₂CH₃)—, and the like can also be used as the branched alkylene group. Among them, a methylene group or an ethylene group is preferable, and a methylene group is more preferable.

In the fluorescent probe of the present invention, it is important that A of General Formula (I) is a proline residue. In the present study, as a result of a screening test using a library of enzyme probes including of about 400 types of HMRG derivative probes, it was found that a fluorescent probe consisting of a dipeptide containing proline which forms an amide bond with HMRG at the C-terminus (XaaP-HMRG) tends to show a significant difference and ratio between an increase of FI in carcinomatous and non-carcinomatous lysates.

As a result of a further screening test, it was found that Xaa (B of General Formula (I)) is an amino acid residue selected from a glycine residue, a glutamic acid residue, a leucine residue, a proline residue, a tyrosine residue, and an N^{α}-acetyl-lysine residue. Furthermore, among these amino acid residues, B is preferably a glycine residue, a leucine residue, a proline residue, a tyrosine residue, or an N^{α}-acetyl-lysine residue, and particularly preferably a glycine residue.

Here, A is linked to adjacent NH in the formula by forming an amide bond, and B is linked to A by forming an amide bond.

Specific examples of the compound of General Formula (I) include the following compounds. However, the present invention is not limited thereto.

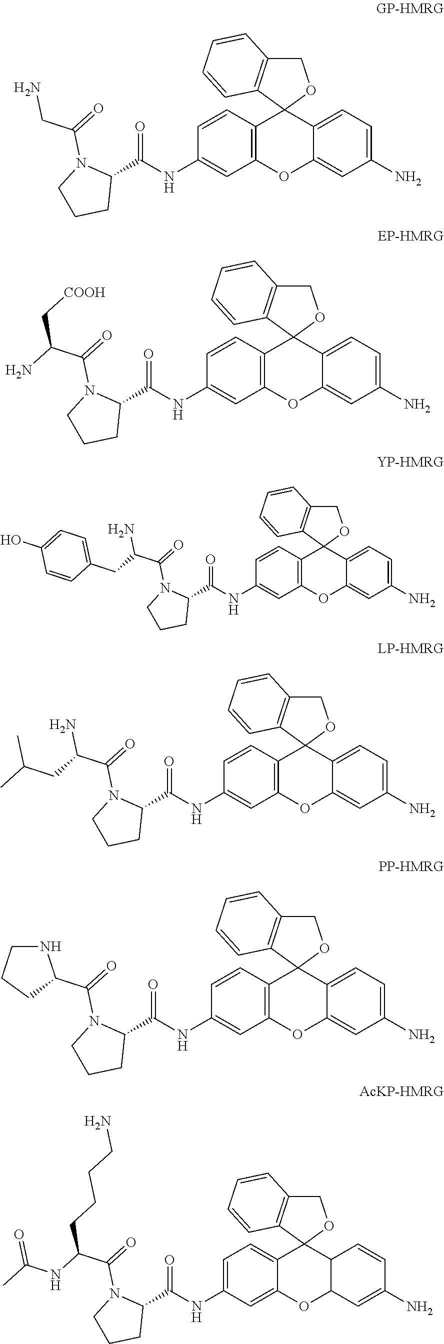

The compound represented by General Formula (I) may exist as a salt. Examples of such a salt include a base addition salt, an acid addition salt, and an amino acid salt. Examples of the base addition salt include metal salts such as a sodium salt, a potassium salt, a calcium salt, and a magnesium salt, an ammonium salt, and organic amine salts such as a triethylamine salt, a piperidine salt, and a morpholine salt, and examples of the acid addition salt include mineral acid salts such as a hydrochloride, a sulfate, and a nitrate, and organic acid salts such as a carboxylate, a methanesulfonate, a paratoluenesulfonate, a citrate, and an oxalate. Examples of the amino acid salt include a glycine salt. However, the present invention is not limited to these salts.

The compound represented by General Formula (I) may have one or two or more asymmetric carbon atoms depending on the type of substituent, and a stereoisomer such as an optical isomer or a diastereoisomer may be present. A stereoisomer in pure form, any mixture of stereoisomers, a racemate, and the like are all encompassed within the scope of the present invention.

The compound represented by General Formula (I) or the salt thereof may also exist as a hydrate or a solvate, and all of these substances are encompassed within the scope of the present invention. The type of solvent for forming the solvate is not particularly limited, and examples thereof include solvents such as ethanol, acetone, and isopropanol.

The compound represented by General Formula (I) can be easily produced, for example, by converting a 2-carboxyphenyl group or a 2-alkoxycarbonylphenyl group at the 9-position into a hydroxyalkyl group and then acylating an amino group at the 3-position using a xanthene compound having an amino group at the 3-position and the 6-position and having a 2-carboxyphenyl group or a 2-alkoxycarbonylphenyl group at the 9-position as a raw material. Examples of a 3,6-diaminoxanthene compound that can be used as a raw material include, but are not limited to, rhodamine 110 and rhodamine 123 both of which are commercially available, and an appropriate xanthene compound can be selected according to a structure of a target compound.

The fluorescent probe of the present invention may be used as a composition by blending an additive usually used for preparation of a reagent, if necessary. For example, as an additive for use in a physiological environment, an additive such as a dissolution aid, a pH adjusting agent, a buffer, or an isotonizing agent can be used, and a blending amount thereof can be appropriately selected by those skilled in the art. These compositions may be provided as compositions in appropriate forms such as a mixture in a powder form, a lyophilizate, a granule, a tablet, and a liquid preparation.

As the fluorescent probe of the present invention, the compound represented by General Formula (I) or the salt thereof may be used as it is, but if necessary, an additive usually used for preparing a reagent may be blended and used as a composition. For example, as an additive for using a reagent in a physiological environment, an additive such as a dissolution aid, a pH adjusting agent, a buffer, or an isotonizing agent can be used, and the blending amount thereof can be appropriately selected by those skilled in the art. These compositions are generally provided as compositions in appropriate forms such as a mixture in a powder form, a lyophilizate, a granule, a tablet, and a liquid preparation, and may be applied by being dissolved in distilled water for injection or an appropriate buffer at the time of use.

Note that the fluorescent probe of the present invention can be used, for example, during surgery, during examination, or after surgery. In the present specification, the term “surgery” includes any surgery including endoscopic surgery such as endoscopic or laparoscopic surgery. In addition, the term “examination” includes an examination performed on tissue separated and collected from a living body, and the like, in addition to an examination using an endoscope and a treatment such as removal and collection of tissue associated with the examination. These terms should be interpreted in the broadest sense and not in any way in a limiting sense.

In addition, in the present specification, the term “carcinomatous tissue” means any tissue including cancer cells. The term “tissue” should be interpreted in the broadest sense to include a part or the whole of an organ, and should not be interpreted in a limiting sense. In addition, in the present specification, the term “diagnosis” should be interpreted in the broadest sense including confirmation of the presence of carcinomatous tissue at any biological site with naked eyes or under a microscope.

One aspect of the present invention is a composition for detecting pancreatic cancer that contains the fluorescent probe of the present invention.

In addition, another aspect of the present invention is a composition for diagnosing pancreatic cancer that contains the fluorescent probe of the present invention.

In addition, still another aspect of the present invention is a composition for diagnosing pancreatic cancer that contains the fluorescent probe of the present invention and is used in a cancer surgical treatment or a cancer examination.

Here, the cancer surgical treatment includes open surgery and endoscopic surgery.

### 3. Detection Method Using Fluorescent Probe

Another embodiment of the present invention is a method for detecting a pancreatic cancer cell or cancer tissue, the method including the steps of:

applying the fluorescent probe of the present invention to tissue collected from a pancreas of a subject;

irradiating the tissue after the application with excitation light; and

detecting fluorescence from the tissue.

Here, the subject includes a human and a mammal other than the human (for example, a dog, a cat, and the like).

In order to apply the fluorescent probe to the tissue collected from the pancreas of the subject in the step (a), for example, the fluorescent probe is applied to, for example, a well of a 384 plate or the like using a lysate prepared from a carcinomatous or non-carcinomatous tissue sample, but the present invention is not limited thereto.

In addition, still another embodiment of the present invention is a method for sensing pancreatic cancer, the method including: (a) a step of applying the fluorescent probe of the present invention to a clinical specimen of pancreatic cancer; and (b) measuring a fluorescence image of the clinical specimen of pancreatic cancer to which the fluorescent probe is applied.

The application of the fluorescent probe to a clinical specimen in the step (a) can be performed, for example, by locally or globally spraying a solution of the fluorescent probe to the clinical specimen.

The detection method and the sensing method of the present invention can further include observing a fluorescence response using a fluorescence imaging means. As a means for observing the fluorescence response, a fluorometer having a wide measurement wavelength can be used, but the fluorescence response can also be visualized using a fluorescence imaging means capable of displaying the fluorescence response as a two-dimensional image. The fluorescence response can be visualized in two dimensions using the fluorescence imaging means, such that a carcinomatous cell or tissue can be instantly visually recognized. As a fluorescence imaging device, a device known in the art can be used. Note that, in some cases, it is also possible to detect the reaction between the sample to be measured and the fluorescent probe by a change in ultraviolet-visible absorption spectrum (for example, a change in absorbance at a specific absorption wavelength).

A method for using the fluorescent probe of the present invention is not particularly limited, and the fluorescent probe can be used in the same manner as a fluorescent probe known in the related art. Usually, it is preferable that the compound of the present invention or the salt thereof is dissolved in an aqueous medium such as physiological saline or a buffer, or a mixture of a water-miscible organic solvent such as ethanol, acetone, ethylene glycol, dimethyl sulfoxide, or dimethylformamide, and an aqueous medium, the solution is added to an appropriate buffer containing cells or tissue, and a fluorescence spectrum is measured. The fluorescent probe of the present invention may be used in the form of a composition in combination with an appropriate additive. For example, the fluorescent probe can be used in combination with an additive such as a buffer, a dissolution aid, or a pH adjusting agent.

In addition, a concentration of the compound of the present invention in the fluorescent probe of the present invention can be appropriately determined according to the type of cells or the like to be measured, measurement conditions, and the like.

Still another embodiment of the present invention is a kit for detecting a pancreatic cancer cell or tissue that contains the fluorescent probe of the present invention.

In the kit, the fluorescent probe of the present invention is generally prepared as a solution, but the fluorescent probe of the present invention can also be provided as, for example, a composition in an appropriate form such as a mixture in a powder form, a lyophilizate, a granule, a tablet, and a liquid preparation and can be dissolved in distilled water for injection or an appropriate buffer at the time of use to be applied.

In addition, the kit may appropriately contain an additional reagent or the like, if necessary. For example, an additive such as a dissolution aid, a pH adjusting agent, a buffer, or an isotonizing agent can be used as the additive, and the blending amount thereof can be appropriately selected by those skilled in the art.

A concentration of the fluorescent probe of the present invention to be applied is not particularly limited, but for example, a solution having a concentration of about 0.1 to 10 μM can be applied.

### 4. Application of Fluorescent Probe of Present Invention to Surgery of Pancreatic Cancer

Still another embodiment of the present invention is a method for determining the presence of a pancreatic cancer cell in a subject and/or identifying a range of pancreatic cancer tissue (hereinafter, also referred to as an “identification method and the like of the present invention”), the method including: (a) a step of applying a fluorescent probe comprising a compound represented by the following General Formula (I) or a salt thereof to a specimen surgically resected from a pancreas of the subject; and (b) measuring a fluorescence image of the resected specimen to which the fluorescent probe is applied.

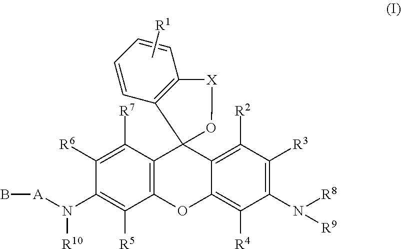

Here, the subject includes a human and a mammal other than the human (for example, a dog, a cat, and the like).

In General Formula (I), R¹, R², R³, R⁴, R⁵, R⁶, R⁷, X, A, and B are as described above.

In the identification method and the like of the present invention, in General Formula (I), it is particularly preferable that B is a glycine residue.

In addition, in the identification method and the like of the present invention, in General Formula (I), it is preferable that R¹, R², R³, R⁴, R⁵, R⁶, R⁷, R⁸, R⁹, and R¹⁰ are hydrogen atoms, and X is a methylene group.

The identification method and the like of the present invention can further include visualizing the fluorescence image using a fluorescence imaging means. Details of the fluorescence imaging means are as described in the detection method and the sensing method of the present invention.

The identification method and the like of the present invention can be performed during a surgical treatment of pancreatic cancer. Here, the surgical treatment of pancreatic cancer includes open surgery and endoscopic surgery.

The identification method and the like of the present invention are performed during a surgical treatment of pancreatic cancer using the fluorescent probe of the present invention, such that it is possible to clearly distinguish carcinomatous tissue from surrounding non-carcinomatous tissue as a fluorescent region in a specimen surgically resected from a pancreas of a subject. In addition, the identification method and the like of the present invention are performed, such that it is possible to identify carcinomatous tissue in a rapid and real time manner based on cancer cell viability. As a result, it is possible to reduce performing a pathological examination of tissue suspected of having cancer during surgery.

In addition, in a patient who received preoperative chemotherapy, cancer invasion that cannot be confirmed by the naked eye may occur around a blood vessel such as the splenic artery, but when the identification method and the like of the present invention are used, it is possible to visualize cancer invasion that cannot be confirmed by the naked eye around a blood vessel such as the splenic artery by fluorescence imaging, which makes it possible to reduce the amount of cancer left during surgery.

As described above, fluorescence imaging by the identification method and the like of the present invention can visualize the spread of living pancreatic cancer cells in real time, which is also useful for intraoperative diagnosis of surgical resection margins and preoperative endoscopic evaluation of luminal lesions.

### Examples

Hereinafter, the present invention will be described in more detail with reference to Examples, but the present invention is not limited to these Examples.

### Materials and Methods

The present study was approved by the Institutional Review Board (IRB No. 2957-[11]) at The University of Tokyo Hospital.

### 1. Collection of Sample

Tissue fragments were collected from resected specimens of patients who underwent radical resection for pancreatic cancer in the period from April 2017 to December 2020. (Informed consent was obtained prior to collection, and consent was obtained)

### 2. Primary Screening

First, according to the procedure described in Non Patent Literatures 6 to 8, each of lysates prepared from tissue fragments of carcinomatous and non-carcinomatous sites was mixed with a fluorescent probe, an increased value of fluorescence intensity was measured, and a fluorescent probe as a candidate was selected. Briefly, 5 μL of a lysate (protein concentration: 0.20 mg/dL) was added dropwise to a well of a black 384 plate in which 15 μL of a library of dipeptide-HMRG compounds (Non Patent Literature 8) was arranged. Final concentrations of the candidate probe and the lysate protein were 1.0 μM and 0.050 mg/dL, respectively.

The fluorescence intensity (FI) of each sample was measured at 0 minutes and 60 minutes after mixing with Envision Multilabel Plate Reader (PerkinElmer, Massachusetts, USA) while performing incubation at 37° C. Excitation and emission wavelengths were set to 485 nm and 535 nm, respectively. An increase in FI was defined as follows.

(Increase in FI)=(FI after 60 minutes)−(FI after 0 minutes)

Probes with the top 10% difference or ratio in the increase in FI in the carcinomatous and non-carcinomatous lysates were selected as candidate probes and evaluated as follows.

### 3. Secondary Screening

Tissue fragments of carcinomatous and non-carcinomatous tissues were placed in an 8-well plate, and the candidate fluorescent probe was directly sprayed. A concentration and amount of each fluorescent probe were 50 μM and 200 μL. Fluorescence images were captured at 0 minutes (before), 1 minute, 3 minutes, 5 minutes, 10 minutes, 15 minutes, 20 minutes, 25 minutes, and 30 minutes after administration of the fluorescent probe. The fluorescence images were acquired with Maestro in Vivo imaging system (PerkinElmer, Massachusetts, USA) and capture conditions were set to excitation and emission wavelengths of 485 to 480 nm and 490 nm. FI was calculated by extracting the fluorescence image at 540 nm and subtracting a fluorescence value of the same region at 1 minute from an average fluorescence value of a region of interest (ROI) at 30 minutes. A fluorescent probe having the largest difference in FI between carcinomatous and non-carcinomatous tissues was selected as a pancreatic cancer-labeled fluorescent probe.

### 4. Macroscopic Evaluation of Cancer Identification Capability Using Surgically Resected Specimen

Immediately after the pancreatic cancer specimen was extracted, the pancreatic cancer specimen was cut so as to include a maximum diameter of pancreatic cancer tissue. Next, the fluorescent probe described above (4 mL, 50 μM) was directly applied to the cut surface and fluorescence imaging was performed using Maestro in Vivo imaging system as described above. The accuracy of the fluorescence imaging was evaluated by a surgeon (RT) and a pathologist (MT) with reference to histopathological findings on the same plane. In addition, a tumor/background ratio (TBR) was also calculated by measuring the increase in average FI from 1 minute to 30 minutes after probe administration in carcinomatous and non-carcinomatous sites based on macroscopic/microscopic findings of the cut surface and corresponding fluorescence images.

### 5. Search for Target Enzyme

It was considered that dipeptidyl peptidase 4 (DPP-IV) or a similar enzyme was a target enzyme overexpressed in pancreatic cancer tissue. First, the candidate fluorescent probe was examined whether DPP-IV or the similar enzyme was activated. Human recombinant DPP-IV (50 μL; D 4943, Sigma-Aldrich), DPP-VIII (1.0 μg; ab162872, abcam), and DPP-IX (1.0 μg; ab79621, abcam) were injected into 3.0 mL of a probe solution (1.0 μM) using F-7000 (Hitachi (Tokyo)), and a change in FI during 2,000 seconds was measured. Excitation and emission wavelengths were set to 495 and 525 nm, respectively.

In addition, expression of DPP-IV in the cut surface of a surgically resected specimen was evaluated by immunohistochemistry (IHC) staining. As an antibody, an anti-DPP-IV mouse monoclonal antibody (TA500733; Origene Technologies Inc. (Rockville, Maryland)) was used. The antigen activation was performed at 110° C. for 15 minutes. A concentration of the anti-DPP-IV antibody was set to 1:100, and incubation was performed at 4° C. overnight. The results of the IHC staining were evaluated by a pathologist (MT) with fluorescence imaging results blinded.

### Example 1

### Primary and Secondary Probe Screenings

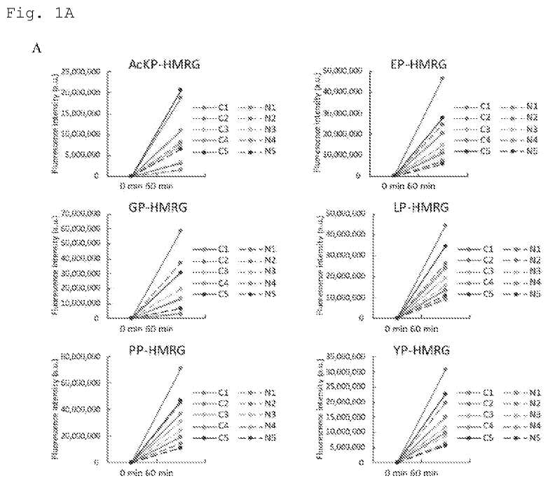

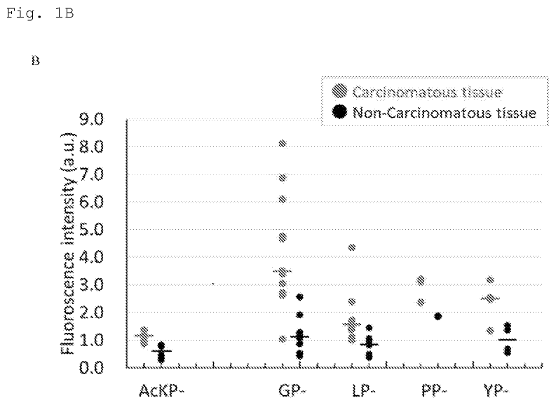

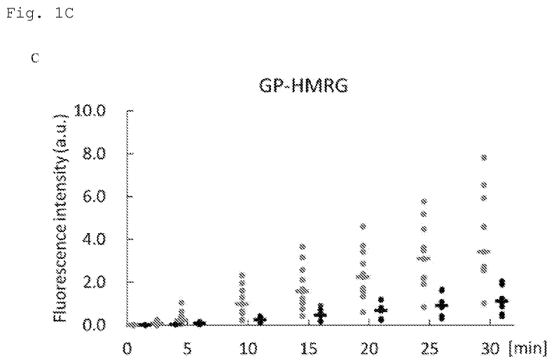

In primary selection using lysates prepared from five resected specimens and 309 candidate probes, a fluorescent probe (XaaP-HMRG) consisting of a dipeptide containing proline forming an amide bond with HMRG at the C-terminus tended to have a large difference or a large increase ratio between increases in FI in carcinomatous and non-carcinomatous lysates (FIG. 1A). Among them, five probes (AcKP-, GP-, LP-, PP-, and YP-HMRG) were selected as candidate probes, and the process proceeds to secondary screening. In the secondary screening, carcinomatous and non-carcinomatous tissue fragments were collected from 11 patients.

FIG. 1 illustrates increases in FI of the candidate probes in the primary and secondary screenings using the lysates and tissue fragments obtained from pancreatic cancer patients.

Specifically, as illustrated in FIG. 1A, fluorescence imaging using lysates from five patients showed a significant increase in FI in carcinomatous tissue (C, solid line) as opposed to non-carcinomatous tissue (N, dotted line) in 6 types of probes (AcKP-, EP-, GP-, LP-, PP-, and YP-HMRG) consisting of a dipeptide containing proline forming an amide bond with HMRG at the C-terminus.

FIG. 1B illustrates an increase in FI at 30 minutes after each probe administration on the tissue fragment. The left plot is the results for the carcinomatous tissue, and the right plot is the results for the non-carcinomatous tissue.

The FI increase ratio of the carcinomatous site/non-carcinomatous site of GP-HMRG was the highest (range, 2.70 to 6.10) among the remaining candidates including AcKP-HMRG (0.95 to 1.36), LP-HMRG (1.10 to 2.38), PP-HMRG (2.37 to 3.20), and YP-HMRG (1.62 to 3.01). The bar indicates a median value.

In addition, FIG. 1C illustrates a change in the increase in FI over time in carcinomatous and non-carcinomatous tissue fragments after administration of GP-HMRG.

As a result, fluorescence imaging using GP-HMRG showed the highest contrast of FI at 30 minutes between carcinomatous and non-carcinomatous tissues (median value [Range], 3.22 [2.60-5.59] a.u. vs. 1.14 [0.86 −1.87] a.u., P=0.010, Wilcoxon's rank-sum test; FIGS. 1B and 1C). From the above, GP-HMRG was selected as a pancreatic cancer-labeled fluorescent probe, and the cancer detectability of the fluorescence imaging was evaluated using the surgically resected specimen.

### Example 2

### Fluorescence Imaging of All Surgical Specimens Using GP-HMRG

Cancer detectability of fluorescence imaging was evaluated by spraying GP-HMRG to the cut surfaces of all surgical specimens immediately after resection of eight pancreatic adenocarcinoma patients. The patient backgrounds were summarized in Table 1. Preoperative adjuvant chemotherapy (NAC) with gemcitabine and nab-paclitaxel was indicated in two patients. Three patients were treated with diabetes, but a DPP-IV inhibitor was not administered before surgery.

TABLE 1
Patient backgrounds and results of fluorescence imaging using GP-HMRG

| | | | | | Preoperative | | | |
|---|---|---|---|---|---|---|---|---|
| | Age | | | NAC | CA19-9 | Surgical | Histological | A TBR |
| Patients | (y) | Sex | DM | (effect*) | (IU/mL) | procedures | type | ratio of FI |
| 1 | 70 | M | − | − | 68 | DP | Adenosquamous | 3.44 |
| 2 | 83 | F | + | − | 55 | DP | tub1 > tub2 | 2.06 |
| 3 | 63 | M | − | − | 9 | PD | tub2 > tub1 > por | 1.98 |
| 4 | 74 | M | − | − | 39 | DP | tub2 > tub1 | 1.98 |
| 5 | 73 | F | + | − | 1 | DP | tub1 > tub2 | 1.93 |
| 6 | 68 | M | + | − | 393 | PD | tub2 > tub1 | 1.47 |
| 7 | 82 | F | − | +, (1b) | 25 | DP-CAR | tub1 > tub2 | 1.26 |
| 8 | 84 | F | − | +, (1b) | 544 | DP | tub1 > tub2** | 1.13 |

Abbreviations and symbols in Table 1 are as follows.

DM: diabetes, NAC: preoperative chemotherapy, PD: pancreatoduodenectomy, DP: distal pancreatectomy, DP-CAR: distal pancreatectomy

pancreatectomy with celiac axis resection: distal pancreatectomy with celiac axis resection

TBR: tumor/background ratio, tub 1/tub 2: well/moderately differentiated tubular adenocarcinoma

adenocarcinoma, por: poorly differentiated adenocarcinoma
*: based on Evans classification
**: invasion of viable cancer cells into perineural and lymphatic vessels into splenic artery

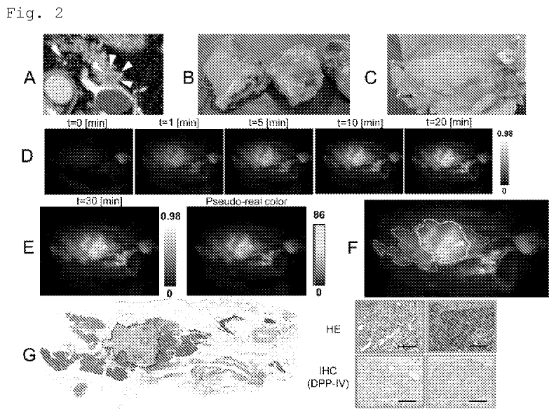

An average TBR of the fluorescence images 30 minutes after GP-HMRG spraying was 1.96 (range, 1.31 to 2.04). In four patients (Patient Nos.: 1, 2, 4, and 5) with a TBR ranging from 1.93 to 3.10, the fluorescence signal of the carcinomatous tissue was almost uniform and macroscopically discernable from the surrounding pancreatic tissue (FIG. 2).

FIG. 2 illustrates fluorescence imaging (Patient No. 2) on all surgical specimens using GP-HMRG showing a uniform increase in fluorescence signal in pancreatic cancer tissue.

A of FIG. 2 illustrates preoperative contrast CT (arrow) of pancreatic body cancer.

B of FIG. 2 illustrates a macroscopic image OR of the DP specimen after creating a cut surface of the tumor, and C is an enlarged view of the cut surface including the tumor.

D of FIG. 2 illustrates an increase in fluorescence signal after spraying GP-HMRG to the cut surface.

E of FIG. 2 illustrates a fluorescence image (left) and a pseudo-real color image (right) of the cut surface 30 minutes after administration of the probe.

F of FIG. 2 illustrates a relationship between the fluorescence signal and the distribution of carcinomatous tissue (white solid line) and surrounding pancreatic tissue (white dotted line) based on the macroscopic image (B) of the sample.

G of FIG. 2 illustrates a low magnification histopathological image of hematoxylin-eosin (H&E) staining corresponding to the fluorescence image (left, dotted line indicates a cancer boundary). An enlarged view of H&E staining and IHC staining of DPP-IV in carcinomatous tissue (red) and pancreatic tissue (blue) is also illustrated (right). The scale bar is 100 μm.

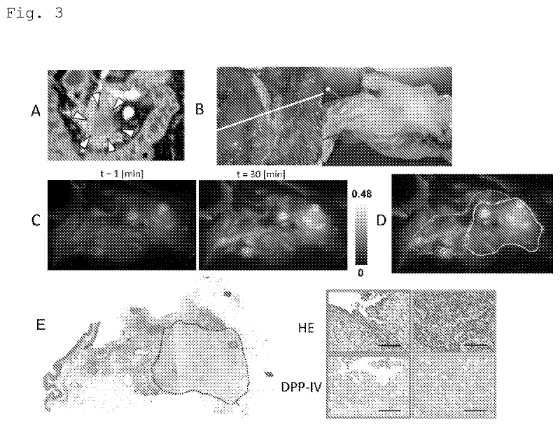

In the remaining four cases including two cases that received preoperative chemotherapy, the carcinomatous tissue showed a non-uniform fluorescence signal, and the fluorescence signal in the carcinomatous tissue was higher than that in the non-carcinomatous tissue, but it was difficult to clearly distinguish between the carcinomatous tissue and the non-carcinomatous tissue (FIG. 3).

FIG. 3 illustrates fluorescence imaging (Patient No. 6) on all surgical specimens using GP-HMRG showing a non-uniform increase in fluorescence signal in pancreatic cancer tissue.

A of FIG. 3 illustrates preoperative contrast CT (arrow) of pancreatic head cancer.

B of FIG. 3 illustrates a macroscopic image of a PD specimen (left) and a cut surface along a dotted line containing a tumor (right).

C of FIG. 3 illustrates an increase in fluorescence signal after spraying GP-HMRG to the cut surface.

D of FIG. 3 illustrates a relationship between the fluorescence signal and the distribution of carcinomatous tissue (white solid line) and surrounding pancreatic tissue (white dotted line) based on the macroscopic image (B) of the sample.

E of FIG. 3 illustrates a low-magnification histopathological image (left, dotted line indicates a cancer boundary) of H&E staining corresponding to the fluorescence image, and also illustrates an enlarged view of H&E staining and IHC staining of DPP-IV in carcinomatous tissue (white solid line) and pancreatic tissue (white dotted line) (right). The scale bar is 100 μm.

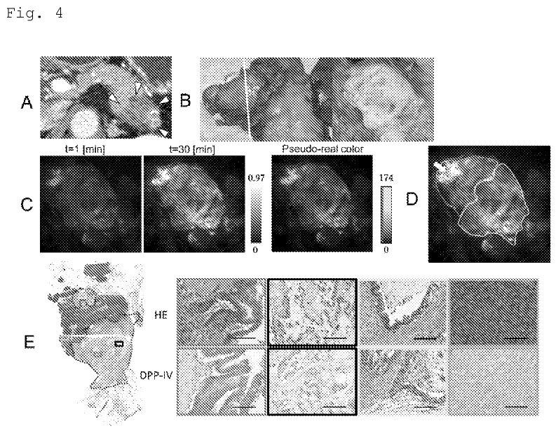

In one patient in the latter group, in fluorescence imaging, a significant increase in signal (a TBR (2.04)) in the connective tissue surrounding the splenic artery was confirmed (arrow in FIG. 4D), and although invasion of viable cancer cells into perineural and lymphatic vessels was diagnosed later on a pathological examination, the main tumor contained fibrosis and mucinous changes (a TBR (1.13)) with few viable cancer cells, likely due to preoperative chemotherapy (FIG. 4).

FIG. 4 illustrates fluorescence imaging (Patient No. 8) on all surgical specimens using GP-HMRG showing cancer invasion into the splenic artery.

A of FIG. 4 illustrates preoperative contrast CT (arrow) of pancreatic body cancer.

B of FIG. 4 illustrates a macroscopic image of a DP specimen (left) and a cut surface along a dotted line containing a tumor (right).

C of FIG. 4 illustrates an increase in fluorescence signal and a pseudo-real color image 30 minutes after spraying GP-HMRG on the cut surface.

D of FIG. 4 illustrates a relationship between the fluorescence signal and the distribution of carcinomatous tissue (white solid line) and surrounding pancreatic tissue (white dotted line) based on the macroscopic image (B) of the sample. The arrow indicates the splenic artery.

E of FIG. 4 illustrates a low-magnification histopathological image (left, dotted line indicates a cancer boundary) of H&E staining corresponding to the fluorescence image. An enlarged view of the fluorescence (red) and a small amount of fluorescence (black) portion of the main tumor by H&E staining and IHC staining of DPP-IV, viable cancer invasion around the splenic artery (green), and non-carcinomatous pancreatic tissue (blue) is also illustrated (right). The scale bar is 100 μm.

### Reference Example 1

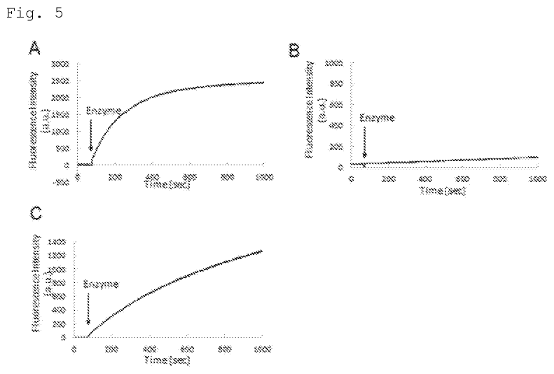

Identification of Target Enzyme Activating GP-HMRG An in vitro fluorescence spectrum of GP-HMRG after addition of DPP showed that the probe was converted into a highly fluorescent HMRG in reaction with DPP-IV and DPP-IX (FIG. 5).

Here, FIG. 5 illustrates a temporal change in fluorescence intensity of GP-HMRG when each enzyme is added. The excitation/emission wavelength was 495 nm/525 nm. A of FIG. 5 illustrates a result of adding DPP-IV, B of FIG. 5 illustrates a result of adding DPP-VIII, and C of FIG. 5 illustrates a result of adding DPP-IX.

On the other hand, contrary to the above results, IHC staining in the resected samples of eight patients showed no apparent difference in DPP-IV expression levels between the carcinomatous tissue and the surrounding pancreatic tissue (Table 1 and FIGS. 2 to 4).

### (Considerations)

In the present study, 309 types of activatable fluorescent probe candidates were screened and GP-HMRG was selected for identification of pancreatic cancer tissue. The fluorescence imaging using GP-HMRG on the cut surfaces of freshly resected specimens visualized carcinomatous tissue as a uniform fluorescent region having a high (>1.9) TBR in four of the eight patients. In the remaining four cases, the fluorescence signal in the carcinomatous tissue was non-uniform, and the fluorescence signal in the carcinomatous tissue was higher than that in the non-carcinomatous tissue, but it was insufficient for clear discrimination from the surrounding non-carcinomatous tissue. However, in one patient who received preoperative chemotherapy, the fluorescence imaging has made it possible to visualize cancer invasion around the splenic artery that could not be confirmed by the naked eye. These results indicate that the fluorescence imaging using GP-HMRG can visualize the spread of viable cancer cells in real time, which is useful for intraoperative diagnosis of surgical resection margins and preoperative endoscopic evaluation of luminal lesions.

The main advantage of using an activatable probe is that it allows rapid and real-time identification of carcinomatous tissue based on enzymatic activity, that is, cancer cell viability. Indeed, in the present study, an increase in fluorescence signal in carcinomatous tissue was confirmed from 1 minute after topical administration of GP-HMRG, and further it was considered that FI in carcinomatous tissue decreased as a result of fibrosis and mucinous changes due to preoperative chemotherapy.

Recently, several studies have also developed fluorescence imaging techniques for the intraoperative identification of pancreatic cancer using a novel fluorophore targeting 5-aminolevulinic acid, indocyanine green, and carbohydrate antigen 19-9 (CA19-9), carcinoembryonic antigen (CEA), epidermal growth factor receptor (EGFR), and insulin-like growth factor 1 receptor (IGF-1R).

However, these techniques based on systemic administration of “deactivatable” probes usually require a longer interval for washout of a fluorescent agent from background tissue, which may result in a lower TBR as compared to the use of activatable probes by intraoperative topical administration. On the other hand, the present technique has a potential advantage in elucidating the viability and enzymatic activity of carcinomatous tissue, and can predict sensitivity to chemotherapy and postoperative outcome.

As described above, in conclusion, fluorescence imaging using GP-HMRG can visualize pancreatic cancer in a rapid and real time manner based on enzymatic activity of carcinomatous tissue.

1. A fluorescent probe for detecting pancreatic cancer, comprising a compound represented by the following General Formula (I) or a salt thereof, 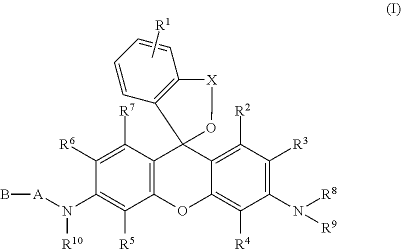
(in the formula,
R¹ represents a hydrogen atom or one to four identical or different substituents bonded to a benzene ring;
R², R³, R⁴, R⁵, R⁶, and R⁷ each independently represent a hydrogen atom, a hydroxyl group, an alkyl group, or a halogen atom;
R⁸, R⁹, and R¹⁰ each independently represent a hydrogen atom or an alkyl group;
X represents a C₁-C₃ alkylene group;
A is a proline residue; and
B is an amino acid residue selected from a glycine residue, a leucine residue, a proline residue, a tyrosine residue, and an N^{α}-acetyl-lysine residue;
wherein A is linked to adjacent NH in the formula by forming an amide bond, and B is linked to A by forming an amide bond).

2. The fluorescent probe according to claim 1, wherein B is a glycine residue.

3. The fluorescent probe according to claim 1, wherein R¹, R², R³, R⁴, R⁵, R⁶, R⁷, R⁸, R⁹, and R¹⁰ are hydrogen atoms, and X is a methylene group.

4. A fluorescent probe for detecting pancreatic cancer, comprising a compound selected from the following group or a salt thereof: 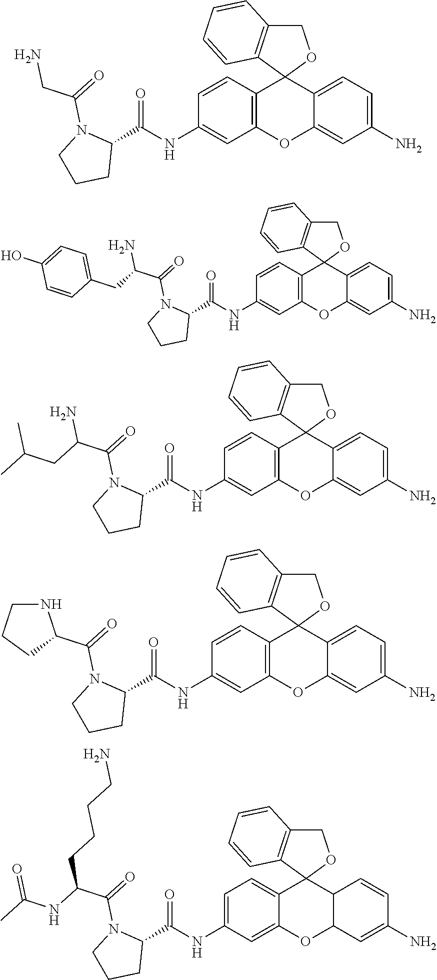
.

5. A composition for detecting pancreatic cancer, the composition comprising the fluorescent probe according to claim 1.

6. A kit for detecting pancreatic cancer, the kit comprising the fluorescent probe according to claim 1.

7. A composition for diagnosing pancreatic cancer, comprising the fluorescent probe according to claim 1.

8. The composition for diagnosing pancreatic cancer according to claim 7, wherein the composition is used in a cancer surgical treatment or a cancer examination.

9. The composition for diagnosing pancreatic cancer according to claim 8, wherein the cancer surgical treatment is open surgery or endoscopic surgery.

10. A method for detecting a pancreatic cancer cell or cancer tissue, the method comprising:
applying the fluorescent probe according to claim 1 to tissue collected from a pancreas of a subject;
irradiating the tissue after the application with excitation light; and
detecting fluorescence from the tissue.

11. A method for sensing pancreatic cancer, the method comprising: (a) a step of applying the fluorescent probe according to claim 1 to a clinical specimen of pancreatic cancer; and (b) measuring a fluorescence image of the clinical specimen of pancreatic cancer to which the fluorescent probe is applied.

12. The method according to claim 11, further comprising visualizing the fluorescence image using a fluorescence imager.

13. A method for determining the presence of a pancreatic cancer cell in a subject and/or identifying a range of pancreatic cancer tissue, the method comprising: (a) applying a fluorescent probe comprising a compound represented by the following General Formula (I) or a salt thereof to a specimen surgically resected from a pancreas of the subject; and (b) measuring a fluorescence image of the resected specimen to which the fluorescent probe is applied, 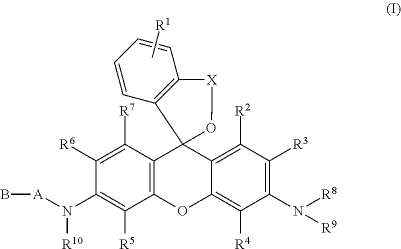
(in the formula,
R¹ represents a hydrogen atom or one to four identical or different substituents bonded to a benzene ring;
R², R³, R⁴, R⁵, R⁶, and R⁷ each independently represent a hydrogen atom, a hydroxyl group, an alkyl group, or a halogen atom;
R⁸, R⁹, and R¹⁰ each independently represent a hydrogen atom or an alkyl group;
X represents a C₁-C₃ alkylene group;
A is a proline residue; and
B is an amino acid residue selected from a glycine residue, a glutamic acid residue, a leucine residue, a proline residue, a tyrosine residue, and an N^{α}-acetyl-lysine residue;
wherein A is linked to adjacent NH in the formula by forming an amide bond, and B is linked to A by forming an amide bond).

14. The method according to claim 13, wherein B is a glycine residue.

15. The method according to claim 13, wherein R¹, R², R³, R⁴, R⁵, R⁶, R⁷, R⁸, R⁹, and R¹⁰ are hydrogen atoms, and X is a methylene group.

16. The method according to claim 13, further comprising visualizing the fluorescence image using a fluorescence imager.

17. The method according to claim 13, wherein said method is performed during a surgical treatment of pancreatic cancer.

18. The method according to claim 17, wherein the surgical treatment of pancreatic cancer is open surgery or endoscopic surgery.
